(19) United States
(12) Patent Application Publication
(10) Pub. No.: US 2024/0081745 A1
(43) Pub. Date: Mar. 14, 2024

(54) DRIVER SUPPORT SYSTEM, DRIVER SUPPORT METHOD, AND STORAGE MEDIUM STORING DRIVER SUPPORT PROGRAM

(71) Applicant: HONDA MOTOR CO., LTD., Tokyo (JP)

(72) Inventors: Hideki Sakai, Wako-shi (JP); Shigenobu Mitsuzawa, Wako-shi (JP); Satoru Shinkawa, Wako-shi (JP); Tomohiro Imai, Wako-shi (JP); Keisuke Nakamura, Wako-shi (JP); Hiroshi Ono, Wako-shi (JP)

(21) Appl. No.: 18/460,230

(22) Filed: Sep. 1, 2023

(30) Foreign Application Priority Data
Sep. 14, 2022 (JP) 2022-146229

(51) Int. Cl. A61B 5/00; A61B 5/024

(52) U.S. Cl. CPC A61B 5/6893; A61B 5/02438; A61B 5/7246

(57) ABSTRACT

A driver support system includes a processor that acquires a heart rate of a target person; evaluates a health condition of the target person based on the heart rate of the target person; and performs an operation setting of a moving object into which the target person gets as a driver. The processor evaluates the health condition of the target person based on an ordinary heart rate, which is a heart rate of the target person during an ordinary period, and a pre-driving heart rate which is a heart rate of the target person when the target person arrives at the moving object, performs the operation setting of the moving object based on a result of the health condition of the target person evaluated when the target person gets into the moving object as a driver.

### INCORPORATION BY REFERENCE

The present application claims priority under 35 U.S.C.§ 119 to Japanese Patent Application No. 2022-146229 filed on Sep. 14, 2022. The content of the application is incorporated herein by reference in its entirety.

### BACKGROUND OF THE INVENTION

### Field of the Invention

The present invention relates to a driver support system, a driver support method, and a non-transitory computer-readable storage medium storing a driver support program.

### Description of the Related Art

A vehicle seat is disclosed in Japanese Patent Laid-Open No. 2019-116279 that is provided with an electrostatic capacitive sensor capable of stably detecting cardiac potential of a seated person.

During diagnosis of a health condition of a person, there may be a case where appropriate diagnosis cannot be performed based only on biometric data of a person who is in a seated state where activities are relatively stable.

An object of the present invention is to appropriately determine a health condition of a driver of a moving object and to provide the driver with appropriate support for safe driving.

### SUMMARY OF THE INVENTION

A first aspect of the present invention provides a driver support system including: an acquisition unit that acquires a heart rate of a target person; an evaluation unit that evaluates a health condition of the target person based on the heart rate of the target person acquired by the acquisition unit; and a setting unit that performs an operation setting of a moving object into which the target person gets as a driver, the evaluation unit being configured to evaluate the health condition of the target person based on an ordinary heart rate, which is a heart rate of the target person during an ordinary period, and a pre-driving heart rate which is a heart rate of the target person when the target person arrives at the moving object, the setting unit being configured to perform the operation setting of the moving object based on a result of the health condition of the target person evaluated by the evaluation unit when the target person gets into the moving object as a driver.

In the driver support system according to the first aspect of the invention, the evaluation unit determines that the health condition of the target person is good when a difference in heart rate obtained by subtracting the ordinary heart rate from the pre-driving heart rate is less than a predetermined threshold value, and determines that the health condition of the target person is poor when the difference in heart rate is equal to or higher than the predetermined threshold value.

In the driver support system according to the first aspect of the invention, the acquisition unit acquires exercise data related to movement exercise of the target person when the target person approaches the moving object, and even when the difference in heart rate is equal to or higher than the predetermined threshold value, the evaluation unit determines that the health condition of the target person is good when determining, based on the exercise data, that the target person approaches the moving object at a speed equal to or higher than a predetermined threshold speed by moving with his/her legs.

In the driver support system according to the first aspect of the invention, even when determining that the difference in heart rate is equal to or higher than the predetermined threshold value and the target person approaches the moving object at the speed equal to or higher than the predetermined threshold speed by moving with his/her legs, the evaluation unit determines that the health condition of the target person is poor when the heart rate of the target person does not decrease within a predetermined range until a predetermined time has elapsed from a start of driving of the moving object.

In the driver support system according to the first aspect of the invention, the setting unit applies a predetermined first setting to the operation setting of the moving object when the evaluation unit determines that the health condition of the target person is good, and applies a predetermined second setting, which is different from the first setting, to the operation setting of the moving object when the evaluation unit determines that the health condition of the target person is poor.

In the driver support system according to the first aspect of the invention, the driver support system further includes a notification unit that performs a notification to the target person who gets into the moving object, and the notification unit performs a first notification, which is a notification for driving support to the target person who is the driver of the moving object when the evaluation unit determines that the health condition of the target person is poor.

In the driver support system according to the first aspect of the invention, the notification unit performs a second notification, which is a notification on the health condition, to the target person when the difference in heart rate is equal to or higher than the predetermined threshold value and the heart rate of the target person does not decrease within a predetermined range until a predetermined time elapses from a start of driving of the moving object.

In the driver support system according to the first aspect of the invention, the driver support system further includes an information transmission unit that transmits position information on a current position of the moving object to a server outside the moving object, and the information transmission unit transmits the position information of the moving object to the server at predetermined time intervals when the difference in heart rate is equal to or higher than the predetermined threshold value and the heart rate of the target person does not decrease within a predetermined range until a predetermined time elapses from a start of driving of the moving object.

In the driver support system according to the first aspect of the invention, the acquisition unit acquires the heart rate of the target person from a wearable terminal attached to a body of the target person.

In the driver support system according to the first aspect of the invention, the acquisition unit acquires, from a heart rate sensor provided on a door handle of the moving object, a heart rate of the target person when the target person takes the door handle, and the evaluation unit uses the heart rate acquired by the acquisition unit from the heart rate sensor provided on the door handle, as the pre-driving heart rate of the target person.

In the driver support system according to the first aspect of the invention, the setting unit limits some of functions of the moving object when the evaluation unit uses, as the pre-driving heart rate, the heart rate acquired from the heart rate sensor provided on the door handle, thereby determining that the health condition of the target person is poor.

A second aspect of the present invention provides a driver support method executed by a computer, the driver support method including: an acquisition step of acquiring a heart rate of a target person; an evaluation step of evaluating a health condition of the target person based on the heart rate of the target person acquired in the acquisition step; and a setting step of performing an operation setting of a moving object into which the target person gets as a driver, the evaluation step including evaluating the health condition of the target person based on an ordinary heart rate, which is a heart rate of the target person during an ordinary period, and a pre-driving heart rate which is a heart rate of the target person when the target person arrives at the moving object, the setting step including performing the operation setting of the moving object based on a result of the health condition of the target person evaluated in the evaluation step when the target person gets into the moving object as a driver.

A third aspect of the present invention provides a driver support program causing a computer of a driver support system to function as: an acquisition unit that acquires a heart rate of a target person; an evaluation unit that evaluates a health condition of the target person based on the heart rate of the target person acquired by the acquisition unit; and a setting unit that performs an operation setting of a moving object into which the target person gets as a driver, the evaluation unit being configured to evaluate the health condition of the target person based on an ordinary heart rate, which is a heart rate of the target person during an ordinary period, and a pre-driving heart rate which is a heart rate of the target person when the target person arrives at the moving object, the setting unit being configured to perform the operation setting of the moving object based on a result of the health condition of the target person evaluated by the evaluation unit when the target person gets into the moving object as a driver.

According to the aspects of the present invention, it is possible to appropriately determine the health condition of the driver of the moving object and to provide the driver with appropriate support for safe driving.

### BRIEF DESCRIPTION OF THE DRAWINGS

FIG. 1 is a diagram showing a configuration of a driver support system according to an embodiment of the present invention;

FIG. 2 is a view showing an example of a configuration of a vehicle equipped with a support device constituting the driver support system, as viewed from a vehicle interior;

FIG. 3 is a diagram showing a configuration of a mobile terminal constituting the driver support system;

FIG. 4 is a diagram showing a configuration of the support device constituting the driver support system; and

FIG. 5 is a flowchart showing a procedure of processing of a driver support method executed by the driver support system.

### DETAILED DESCRIPTION OF THE PREFERRED EMBODIMENT

### Embodiment

An embodiment of the present invention will be described below with reference to the drawings.

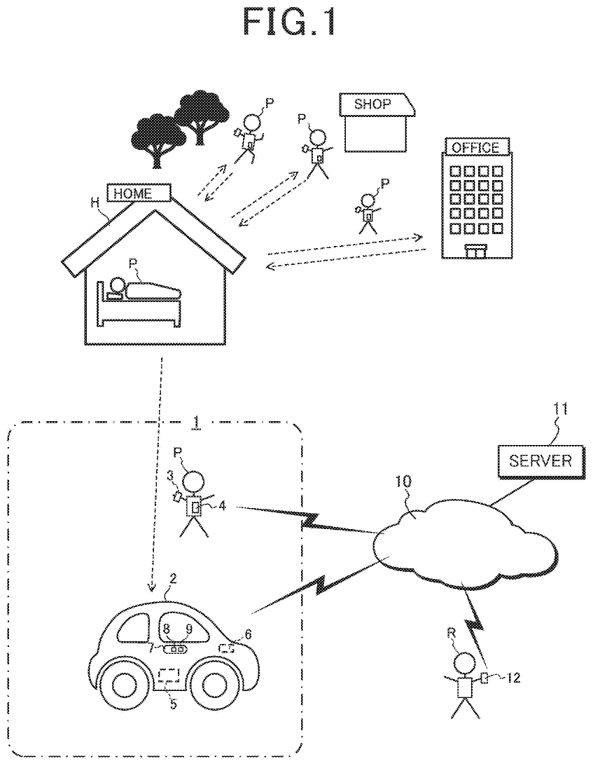

FIG. 1 is a diagram for explaining a configuration of a driver support system 1 according to an embodiment of the present invention together with an example of its use scene. When a target person P gets in a vehicle 2, which is a moving object, the driver support system 1 evaluates health condition of the target person P.

The driver support system 1 includes a wearable terminal 3 and a mobile terminal 4 used by the target person P, and a support device 5 mounted on the vehicle 2 used by the target person P as a driver. By short-range communication such as Bluetooth (registered trademark), the wearable terminal 3 and the mobile terminal 4 are communicably connected to each other, and the mobile terminal 4 and the support device 5 are communicably connected to each other. Further, the mobile terminal 4 and the support device 5 are communicably connected to a server 11 outside the vehicle 2 via a communication network such as the Internet. The vehicle 2 includes a vehicle control device 6 having a processor that controls a door locking operation and a traveling operation of the vehicle 2. The vehicle control device 6 realizes, as control of traveling operation, an ADAS (Advanced Driver-Assistance System) function such as a front vehicle follow-up traveling function or an automatic braking function. In the present disclosure, the vehicle control device 6 is an example of an in-vehicle device that causes the driver support system 1 to set the operation of the vehicle 2. The server 11 is configured with a computer.

The mobile terminal 4 is, for example, a smartphone, and includes a camera, an acceleration sensor, and/or a GNSS (Global Navigation Satellite System) sensor. The wearable terminal 3 is, for example, a wristwatch terminal device, and includes various sensors of a pulse wave sensor, a heart rate sensor, a blood pressure sensor, acceleration sensor, and/or a GNSS sensor, for example, according to the related art, thereby measuring biometric data of the target person P and transmitting the measured biometric data to the mobile terminal 4.

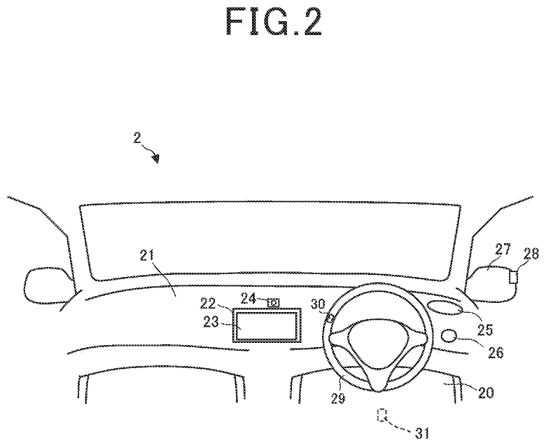

FIG. 2 is a view showing an example of a configuration of the vehicle 2 as viewed from a vehicle interior. An instrument panel 21 in front of a driver's seat 20 is provided with AV (Audio-Visual) device 22 and an interior camera 24 which are arranged approximately in a center in a vehicle width direction. The AV device 22 is, for example, a display/audio device, and displays images and videos (hereinafter, simply referred to as images) on a first touch panel 23 and acquires information input by a touch operation on the first touch panel 23. In addition, the AV device 22 outputs a voice and receives a voice input from a speaker 25 and a microphone 26 provided closer to the driver's seat on the instrument panel 21. The interior camera 24 is, for example, a DMC (Driver Monitoring Camera), and captures an image of the vehicle interior including an image of the driver.

Further, the vehicle 2 includes an exterior camera 28 provided in a door mirror 27 on a side closer to the driver's seat 20. The exterior camera 28 captures an image of a surrounding environment outside the right side of the vehicle 2. For example, the exterior camera 28 captures an image of a person approaching a vehicle door (not shown) on the driver's seat 20 from the right outside of the vehicle 2. The exterior camera 28 is not limited to one, and may configured by a plurality of cameras distributed over the body of the vehicle 2 to capture a whole or part of the surrounding environment of the vehicle 2.

In the vehicle 2, a second heart rate sensor 30 may be provided on a steering wheel 29, and a seating sensor 31 may be provided on the driver's seat 20 to detect seating of a person.

It should be understood FIG. 1 that the display of a plurality of target persons P shows the same target person P in various activity scenes. The wearable terminal 3 is worn on the arm of the target person P during all or most of the day, and measures uninterruptedly or chronologically continuously biometric data of the target person P various daily activities including sleeping, eating, and the like at home, exercising (for example, walking, running, and physical training at the gym), shopping, and commuting at predetermined time intervals. The wearable terminal 3 transmits the measured biometric data to the mobile terminal 4, and the mobile terminal 4 receives and acquires such biometric data.

Then, the mobile terminal 4 calculates and stores an ordinary heart rate, which is a heart rate of the target person P during an ordinary period, from the biometric data of the target person P in the daily activities received from the wearable terminal 3. For example, the mobile terminal 4 calculates and stores, based on the heart rate of the biometric data for the most recent predetermined number of days (for example, one month), the ordinary heart rate every predetermined number of days.

In the present embodiment, particularly, the mobile terminal 4 evaluates the health condition of the target person P, based on the ordinary heart rate and a pre-driving heart rate, which is a heart rate of the target person P when the target person P arrives at the vehicle 2 to get into the vehicle as a driver. Then, when the target person P gets into the vehicle 2 as the driver, the mobile terminal 4 instructs the support device 5 to set an operation of the vehicle, based on the result of the evaluation of the health condition of the target person P.

For example, when a difference in heart rate obtained by subtracting the ordinary heart rate from the pre-driving heart rate is less than a predetermined threshold value, the mobile terminal 4 determines that the health condition of the target person P is good, and when the difference in heart rate is equal to or higher than the predetermined threshold value, the mobile terminal 4 determines that the health condition of the target person P is poor.

In the present embodiment, particularly, the mobile terminal 4 considers exercise data related to moving exercise of the target person P who is approaching the vehicle 2, such that a normal increase in heart rate due to the exercise state of the target person P until arriving at the vehicle 2 (for example, an increase in heart rate due to running) is not treated as a sign of poor health.

In other words, the mobile terminal 4 uses, as exercise data, for example, a speed of change in position of the mobile terminal 4 and a speed of change in number of steps counted, whereby even when the difference in heart rate is equal to or higher than the predetermined threshold value, the mobile terminal 4 determines that the health condition of the target person P is good in a case of determining based on the exercise data that the target person P approaches the vehicle 2 at a speed equal to or higher than a predetermined threshold speed by moving with his/her legs.

However, when the heart rate of the target person P does not restore to near the ordinary heart rate even after the target person P starts to drive the vehicle 2, the health condition is considered to be poor. For this reason, even when determining that the health condition of the target person P is good in consideration of the exercise data as described above, the mobile terminal 4 determines that the health condition of the target person P is poor when the heart rate of the target person P does not decrease within a predetermined range until a predetermined time has elapsed since the start of driving of the vehicle 2.

Then, when the target person P gets into the vehicle 2 as the driver, the mobile terminal 4 cooperates with the support device 5 of the vehicle 2, and performs operation setting of the vehicle 2 based on the evaluation result on the health condition of the target person P (for example, the result of determination as to whether the health condition is good or poor).

Specifically, when it is determined that the health condition of the target person is good, the mobile terminal 4 and the support device 5 set the operation setting of the vehicle 2 to a predetermined first setting, and when it is determined that the health condition of the target person P is poor, the mobile terminal 4 and the support device 5 change the operation setting of the vehicle 2 to a predetermined second setting different from the first setting. For example, the second setting can be a safety setting with more safety considerations than the first setting. Specifically, for example, the support device 5 cooperating with the mobile terminal 4 sets an inter-vehicle distance in the front vehicle follow-up traveling executed by the vehicle control device 6 provided in the vehicle 2 to be longer in the second setting rather than the first setting. Alternatively, the support device 5 sets a start condition of the automatic braking executed by the vehicle control device 6 provided in the vehicle 2 to be decelerated earlier in the second setting rather than the first setting.

Here, the mobile terminal 4 can use, as the pre-driving heart rate, the heart rate acquired from a first heart rate sensor 9 provided on a door handle 7 on an outer surface of the vehicle 2. The mobile terminal 4 uses, as the pre-driving heart rate, the heart rate obtained from the first heart rate sensor 9, whereby when determining that the health condition of the target person P is poor, the mobile terminal 4 cooperates with the support device 5 and instructs the vehicle control device 6 to limit some of functions of the vehicle 2. For example, the mobile terminal 4 can prevent the target person P from driving the vehicle 2 by prohibiting the door lock of the vehicle 2 from being released or prohibiting the vehicle 2 from being powered on.

In addition, when determining that the health condition of the target person P is poor, the mobile terminal 4 performs a first notification, which is a notification for driving support to the target person. At this time, the first notification may be, for example, a notification of calling attention to the target person P who is the driver, such as “You are out of breath. Please take a breath before driving” or a notification of advice on condition recovery. The mobile terminal 4 can perform the notification from, for example, a touch panel or a speaker provided in the mobile terminal 4, or from the first touch panel 23 and/or the speaker 25 in the vehicle by instructing the support device 5 of the vehicle 2 to output a text message and/or a voice message.

Thus, the driver support system 1 can give appropriate advice on driving of the vehicle 2 to the target person P when the health condition is not good.

When the above-described difference in heart rate is equal to or higher than the predetermined threshold value and the heart rate of the target person P does not decrease within a predetermined range until a predetermined time elapsed from the start of driving of the vehicle 2, the mobile terminal 4 performs the second notification, which is a notification on the health condition, to the target person P. The second notification may be, for example, an announcement and a notification of calling attention about the current health condition such as “Your heart rate remains high. Please be careful”.

Further, as described above, when the above-described difference in heart rate is equal to or higher than the predetermined threshold value and the heart rate of the target person P does not decrease within a predetermined range until a predetermined time elapses from the start of driving of the vehicle 2, the mobile terminal 4 instructs the support device 5 to transmit position information on the current position of the vehicle 2 to the server 11. Thus, according to the driver support system 1, a related person R such as a family member or an acquaintance of the target person can access the server 11 using own mobile terminal 12 and watch movement of the target person P.

Instead of instructing the support device 5 to transmit the position information of the vehicle 2 to the server 11, the mobile terminal 4 may receive the position information of the vehicle 2 from the support device 5 at predetermined time intervals and transmit the position information to the server 11.

Configurations of the mobile terminal 4 and the support device 5 will be described below.

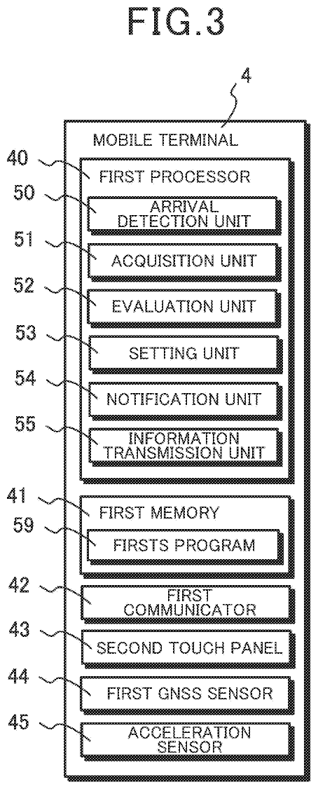

FIG. 3 is a diagram showing a configuration of the mobile terminal 4 constituting the driver support system 1.

The mobile terminal 4 is, for example, a smartphone. The mobile terminal 4 includes a first processor 40, a first memory 41, a first communicator 42 having a transmitter and a receiver, a second touch panel 43, a first GNSS sensor 44, and an acceleration sensor 45. The acceleration sensor 45 is, for example, a 9-axis acceleration sensor.

The first memory 41 may be configured by volatile and/or non-volatile semiconductor memory. The first communicator 42 is a transceiver through which the mobile terminal 4 communicates with the wearable terminal 3, the support device 5, and/or the server 11. For example, the first communicator 42 can include a short-range communicator for performing short-range communication conforming to a Bluetooth (registered trademark) communication standard between the wearable terminal 3 and the support device 5 of the vehicle 2 and a long-range communicator for communicating with the server 11 via the communication network 10.

The first processor 40 is a computer including a CPU (Central Processing Unit), for example. The first processor 40 may include a ROM (Read Only Memory) in which a program is written, a RAM (Random Access Memory) for temporarily storing data, and the like. Then, the first processor 40 includes a arrival detection unit 50, an acquisition unit 51, an evaluation unit 52, a setting unit 53, a notification unit 54, and an information transmission unit 55, as functional components or functional units.

These functional components of the first processor 40 are implemented when the first processor 40 serving as a computer executes a first program 59 stored in the first memory 41. The first program 59 can be stored in any computer-readable storage medium. Alternatively, all or some of the functional components of the first processor 40 may be configured by hardware including one or more electronic circuit components. Here, the first program 59 constitutes a driver support program in the present disclosure together with a second program 79 executed by the support device 5.

The arrival detection unit 50 detects that the target person P arrives at the vehicle 2. Whether the target person P arrives at the vehicle can be determined based on the position information obtained from the first GNSS sensor 44 and the position information of the vehicle 2. The position information of the vehicle 2 can be acquired from the support device 5 via Bluetooth communication established between the mobile terminal 4 and the support device 5 of the vehicle 2 via the first communicator 42. Alternatively, the arrival detection unit 50 may calculate a distance between the mobile terminal 4 and the vehicle 2 from the received radio field intensity of the Bluetooth communication established with the support device 5, and may determine that the target person P arrives at the vehicle 2 when the calculated distance is within a predetermined distance (for example, within 1 m). Alternatively, the arrival detection unit 50 may determine that the target person P arrives at the vehicle 2 when the target person P touches the touch sensor 8 provided on the door handle 7 of the vehicle 2. Information as to whether the touch sensor 8 is touched can be acquired from the support device 5 via the Bluetooth communication.

The acquisition unit 51 acquires a heart rate of the target person P. For example, the acquisition unit 51 acquires the heart rate of the target person P from the wearable terminal 3 worn by the target person P. In addition, the acquisition unit 51 acquires an ordinary heart rate which is a heart rate of the target person P during an ordinary period. For example, the acquisition unit 51 can calculate a frequency distribution of the heat rate of the target person P during daytime obtained from the wearable terminal 3, and can acquire the most frequent heart rate as an ordinary heart rate. Alternatively, the acquisition unit 51 may calculate and acquire, as an ordinary heart rate, an average heart rate of the target person P obtained from the wearable terminal 3 or the heart rate before the target person P leaves home (for example, an average value of the heart rate within a predetermined time before departure) among heart rates obtained from the wearable terminal 3. The time when the target person P leaves the home can be specified from the time when the target person P leaves the position of the home, for example, based on the position information acquired from the first GNSS sensor 44 at predetermined time intervals.

The acquisition unit 51 may also acquire the heart rate of the target person P detected by the first heart rate sensor 9 provided on the door handle 7 via the support device 5 when the target person P takes the door handle 7 of the vehicle 2.

The acquisition unit 51 further acquires exercise data related to movement exercise of the target person P when approaching the vehicle 2. The exercise data includes at least information as to whether the target person is moving using his/her legs and information about a movement speed of the target person P. For example, the acquisition unit 51 can count, based on the time change of the acceleration obtained from the acceleration sensor 45, the number of steps according to the related art, and when the number of steps is counted up, the acquisition unit 51 can determine that the target person moves using his/her legs. In addition, the acquisition unit 51 can calculate the movement speed of the target person P based on the position information acquired from the first GNSS sensor 44 at predetermined time intervals, for example. Alternatively, the acquisition unit 51 may calculate the movement speed of the target person P based on a speed of counting of the number of steps (for example, the number of steps counted per second) and an average step width of a person (for example, an adult).

The evaluation unit 52 evaluates the health condition of the target person P based on the heart rate of the target person P acquired by the acquisition unit 51. In the present embodiment, particularly, the evaluation unit 52 evaluates the health condition of the target person P based on the ordinary heart rate of the target person P and the pre-driving heart rate, which is the heart rate of the target person P when the target person P arrives at the vehicle 2. The evaluation unit 52 can set, as the pre-driving heart rate, the heart rate of the target person P acquired by the acquisition unit 51 when the arrival detection unit 50 detects that the target person P arrives at the vehicle 2. Alternatively, the evaluation unit 52 may use, as the pre-driving heart rate, the heart rate acquired by the acquisition unit 51 from the first heart rate sensor 9 provided on the door handle 7.

The evaluation unit 52 determines that the health condition of the target person P is good, for example, when the difference in heart rate, which is a difference between the pre-driving heart rate and the ordinary heart rate, is less than the predetermined threshold value, and the evaluation unit 52 determines that the health condition of the target person P is poor when the difference in heart rate is equal to or higher the predetermined threshold value.

However, in order not to treat, as a sign of poor health, a normal increase in heart rate due to the exercise state of the target person P until arriving at the vehicle 2 (for example, an increase in heart rate due to running), the evaluation unit 52 determines the health condition of the target person P in consideration of the exercise data related to the movement exercise of the target person P who is approaching the vehicle 2 when the difference in heart rate is equal to or higher than the predetermined threshold value.

Specifically, even when the difference in heart rate is equal to or higher than the predetermined threshold value, the evaluation unit 52 determines that the health condition of the target person P is good in a case of determining, based on the exercise data acquired by the acquisition unit 51, that the target person P approaches the vehicle 2 at a speed equal to or higher than a predetermined threshold speed by moving with his/her legs.

In this case, when the actual heart condition of the target person P is poor, the heart rate of the target person P may not restore to near the ordinary heart rate even after the target person P starts to drive the vehicle 2, and the pre-driving heart rate may be maintained in a state of being higher than that in the ordinary period. For this reason, even when it is determined that the difference in heart rate is equal to or higher than the predetermined threshold value and the target person P approaches the vehicle 2 at a speed equal to or higher than the predetermined threshold speed by moving with his/her legs, the evaluation unit 52 determines that the health condition of the target person P is poor when the heart rate of the target person P does not decrease within a predetermined range (for example, within a range of 1.1 times or less of the ordinary heart rate) until a predetermined time has elapsed since the start of driving of the vehicle 2. Here, the evaluation unit 52 can acquire, from the support device 5, information as to whether the vehicle 2 starts traveling (for example, information as to whether the vehicle speed is equal to or higher than a predetermined speed), and can determine that the target person P start to drive when the vehicle 2 starts traveling.

The setting unit 53 performs the operation setting of the vehicle 2 into which the target person P gets as a driver. In the present embodiment, particularly, the setting unit 53 cooperates with the support device 5, and performs the operation setting of the vehicle 2 based on the evaluation result on the health condition of the target person P in the evaluation unit 52 (for example, the result of determination as to whether the health condition is good or poor) when the target person P gets into the vehicle 2 as a driver.

Specifically, when the evaluation unit 52 determines that the health condition of the target person P is good, the setting unit 53 instructs the support device 5 to apply the predetermined first setting to the operation setting of the vehicle 2. In addition, when the evaluation unit 52 determines that the health condition of the target person P is poor, the setting unit 53 instructs the support device 5 to apply the predetermined second setting different from the first setting to the operation setting of the vehicle 2. As described above, the second setting can be, for example, a safety setting with more safety considerations than the first setting. Thus, it is possible to switch the operation setting of the vehicle 2 according to the health condition of the target person P at the start of driving, and to provide the target person P with appropriate support for safe driving.

Here, specific contents of the first setting and the second setting can be determined in advance in the support device 5 according to vehicle functions provided in the vehicle 2. As described above, according to the present embodiment, as an example, the support device 5 sets the inter-vehicle distance in the front vehicle follow-up traveling executed by the vehicle control device 6 provided in the vehicle 2 to be longer in the second setting rather than the first setting. Further, the support device 5 sets the start condition of the automatic braking executed by the vehicle control device 6 provided in the vehicle 2 to be decelerated earlier in the second setting rather than the first setting.

When the evaluation unit 52 determines that the health condition of the target person P is poor using, as the pre-driving heart rate, the heart rate acquired from the first heart rate sensor 9 provided on a door handle 7, the setting unit 53 cooperates with the support device 5 and limits some of functions of the vehicle 2 with the vehicle control device 6. As described above, for example, the mobile terminal 4 can prevent, via the support device 5, the target person P from driving the vehicle 2 by causing the vehicle control device 6 to prohibit the door lock of the vehicle 2 from being released or prohibit the vehicle 2 from being powered on. Alternatively, the setting unit 53 limits the ADAS function executed by the vehicle control device 6, via the support device 5. Examples of such a limit may include limiting a lower limit value of the inter-vehicle distance in the front vehicle follow-up traveling executed by the vehicle control device 6 to a value larger than a default lower limit value and/or limiting the start condition of the automatic braking executed by the vehicle control device 6 to be decelerated earlier than a default setting.

Thus, it is possible to limit the operation of the vehicle 2 when the target person P whose health condition is poor gets into the vehicle 2, thereby preventing accidents beforehand and avoiding dangerous traveling.

The notification unit 54 performs a notification to the target person P who gets into the vehicle 2. In the present embodiment, particularly, when the evaluation unit 52 determines that the health condition of the target person P is poor, the notification unit 54 performs the first notification, which is a notification for driving support to the target person P who is the driver of the vehicle 2. The first notification may be, for example, a notification of calling attention to the target person P who is the driver, such as “You are out of breath. Please take a breath before driving” or a notification of advice on condition recovery. Alternatively, the first notification may be a notification that encourages active rest, or a notification for notifying that, when the vehicle 2 is about to pass through a location where a traffic accident occurred in the past, the location is a traffic accident occurrence point.

Thus, according to the driver support system 1, for example, since the health condition is notified only when the health condition is not good, it is possible to avoid bothering the target person P with unnecessary notifications about the health condition. Further, according to the driver support system 1, for example, since the target person P is notified when the health condition is not good, the target person P can objectively grasp his/her health condition at an appropriate timing. Therefore, according to the driver support system 1, highly persuasive support and advice can be given to the target person P, such as prompting the target person P to make an appropriate decision about starting to drive the vehicle 2.

When the difference in heart rate calculated by the evaluation unit 52 is equal to or higher than the predetermined threshold value and the heart rate of the target person P does not decrease within a predetermined range (for example, within a range of 1.1 times or less of the ordinary heart rate) until a predetermined time elapses from the start of driving of the vehicle 2, the notification unit 54 performs the second notification, which is a notification on the health condition, to the target person P. As described above, the second notification may be, for example, an announcement and a notification of calling attention about the current health condition such as “Your heart rate remains high. Please be careful”.

The notification unit 54 can make the notification by, for example, instructing the support device 5 of the vehicle 2 to output a text message and/or a voice message from the first touch panel 23 and/or the speaker 25 mounted on the vehicle. Alternatively, the notification unit 54 may perform the notification using the touch panel or the speaker normally provided in the mobile terminal 4.

The information transmission unit 55 transmits position information on the current position of the vehicle 2 to the server 11 outside the vehicle 2 at predetermined time intervals. The position information can be transmitted by a position information transmission unit 75 (to be described below) of the support device 5 when the information transmission unit 55 instructs the support device 5 of the vehicle 2. Alternatively, the information transmission unit 55 may receive the position information of the vehicle 2 from the support device 5 at predetermined time intervals or acquire the position information at predetermined time intervals from the first GNSS sensor 44 provided in the mobile terminal 4, and may transmit the position information to the server 11.

In the present embodiment, particularly, when the difference in heart rate calculated by the evaluation unit 52 is equal to or higher than the predetermined threshold value and the heart rate of the target person P does not decrease within a predetermined range (for example, within a range of 1.1 times or less of the ordinary heart rate) until a predetermined time elapses from the start of driving of the vehicle 2, the information transmission unit 55 transmits the position information.

Thus, according to the driver support system 1, the related person R such as a family member or an acquaintance of the target person can access the server 11 using own mobile terminal 12 to trace whereabouts or movement routes of the target person P who is determined to be poor in the health condition, thereby watching movement of the target person. In addition, since the position information is transmitted when the evaluation unit 52 determines that the health condition of the target person P is poor (for example, only in such a case), the privacy of the target person P can be protected in a normal case where the health condition is good.

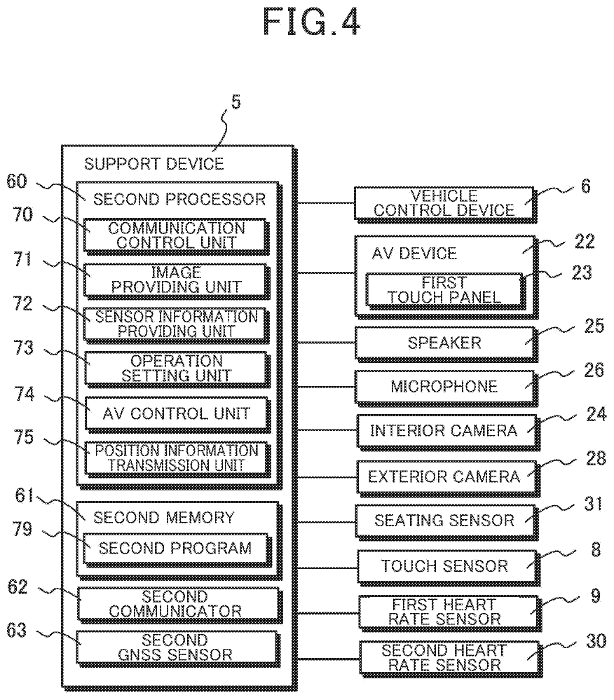

FIG. 4 is a diagram showing a configuration of the support device 5 provided in the vehicle 2.

The support device 5 includes a second processor 60, a second memory 61, a second communicator 62 having a transmitter and a receiver, and a second GNSS sensor 63.

The second memory 61 may be configured by volatile and/or non-volatile semiconductor memory. The second communicator 62 is a transceiver through which the support device 5 communicates with the mobile terminal 4, the server 11, and/or the in-vehicle devices such as the vehicle control device 6. For example, the second communicator 62 can include a short-range communicator for performing short-range communication conforming to a Bluetooth communication standard with the mobile terminal 4, a long-range communicator for communicating with the server 11 via the communication network 10, and a network communicator for performing communication with various in-vehicle devices such as the vehicle control device 6 via an in-vehicle network.

The second processor 60 is a computer including a CPU, for example. The second processor 60 may include a ROM in which a program is written, a RAM for temporarily storing data, and the like. The second processor 60 includes a communication control unit 70, an image providing unit 71, a sensor information providing unit 72, an operation setting unit 73, an AV control unit 74, and a position information transmission unit 75, as functional components or functional units.

These functional components of the second processor 60 are implemented when the second processor 60 serving as a computer executes a second program 79 stored in the second memory 61. The second program 79 can be stored in any computer-readable storage medium. Alternatively, all or some of the functional components of the second processor 60 may be configured by hardware including one or more electronic circuit components. As described above, the second program 79 constitutes the driver support system in the present disclosure together with the first program 59 executed by the mobile terminal 4. Here, the first program 59 constitutes the driver support program in the present disclosure together with the second program 79 executed by the support device 5.

The communication control unit 70 controls and monitors a communication operation of the second communicator 62. In the present embodiment, particularly, the communication control unit 70 detects that the second communicator 62 establishes a Bluetooth communication connection with the mobile terminal 4.

Upon receiving a request received from the mobile terminal 4, the image providing unit 71 transmits an image of the interior of the vehicle 2 captured by the interior camera 24 and/or an image of the surroundings of the vehicle 2 captured by the exterior camera 28, to the mobile terminal 4.

Upon receiving a request received from the mobile terminal 4, the sensor information providing unit 72 transmits, to the mobile terminal 4, information about the fact that the person is seated in the driver's seat 20, the fact that the person touches the touch sensor 8, the heart rate of the person who touches the first heart rate sensor 9, and/or the heart rate of the person who touches the second heart rate sensor 30, based on sensor signals from the seating sensor 31, the touch sensor 8, the first heart rate sensor 9, and/or the second heart rate sensor 30.

The operation setting unit 73 performs the operation setting of the vehicle 2. In the present embodiment, as an example, the operation setting unit 73 applies the predetermined first setting or second setting to the operating setting of the vehicle control device 6 provided in the vehicle 2 according to an instruction from the mobile terminal 4.

As described above, the second setting may be a safety setting with more safety considerations than the first setting. In the present embodiment, the inter-vehicle distance in the front vehicle follow-up traveling executed by the vehicle control device 6 is set to be longer in the second setting rather than the first setting. Alternatively or additionally, the start condition of the automatic braking executed by the vehicle control device 6 is set to be decelerated earlier in the second setting rather than the first setting.

The AV control unit 74 causes the AV device 22 to output image information and voice information through the first touch panel 23 and the speaker 25. In addition, the AV control unit 74 receives and processes the operation input from the first touch panel 23 and the voice input from the microphone 26. In the present embodiment, particularly, the AV control unit 74 causes the AV device 22 to output the notification to the target person P described above according to the instruction received from the mobile terminal 4. Such an output may be an image output such as a text to the first touch panel 23 and/or a voice output from the speaker 25.

Upon receiving the instruction from the mobile terminal 4, the position information transmission unit 75 acquires the position information indicating the current position of the vehicle 2 from the second GNSS sensor 63 at predetermined time intervals, and transmits the position information to the server 11. Further, upon receiving the request from the mobile terminal 4, the position information transmission unit 75 transmits the position information indicating the current position of the vehicle 2 to the mobile terminal 4.

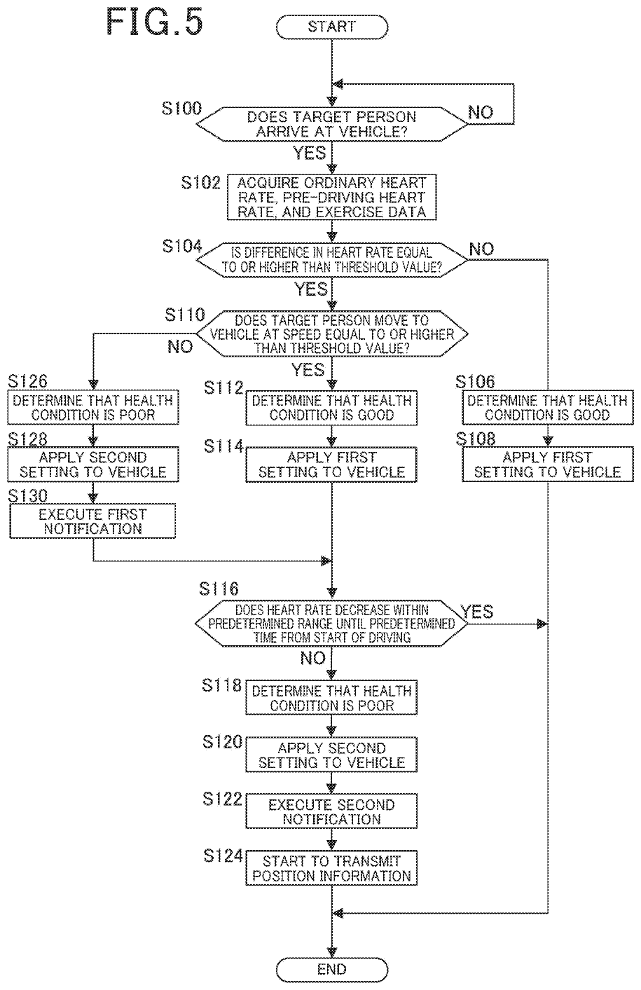

Next, a procedure of the operation in the driver support system 1 will be described. FIG. 5 is a flowchart showing a procedure of processing of a driver support method executed by the driver support system 1. The processing in FIG. 5 is repeatedly executed. In parallel with this processing, the acquisition unit 51 of the mobile terminal 4 executes an acquisition step of acquiring the heart rate of the target person P from the wearable terminal 3 or the like as a part of the driver support method.

When the processing starts, first, the arrival detection unit 50 of the mobile terminal 4 constituting the driver support system 1 determines whether the target person P carrying the mobile terminal 4 arrives at the vehicle 2 (S100). Then, when the target person P does not arrive at the vehicle 2 (S100, NO), the process returns to step S100, and the arrival detection unit 50 repeats the process and waits for the target person P to arrive at the vehicle 2.

On the other hand, when the target person P arrives at the vehicle 2 (S100, YES), the evaluation unit 52 acquires the ordinary heart rate, the pre-driving heart rate, and the exercise data of the target person P until the target person P arrives at the vehicle 2 (S102). Next, the evaluation unit 52 calculates a difference in heart rate by subtracting the ordinary heart rate from the pre-driving heart rate, and determines whether the calculated difference in heart rate is equal to or higher than a predetermined threshold value (S104).

Then, when the difference in heart rate is less than the threshold value (S104, NO), the evaluation unit 52 determines that the health condition of the target person P is good (S106), and the setting unit 53 instructs the support device 5 of the vehicle 2 to apply the first setting to the operation of the vehicle 2 (S108). Thereafter, the setting unit 53 ends this processing.

On the other hand, when the difference in heart rate is equal to or higher than the threshold value in step S104 (S104, YES), the evaluation unit 52 determines, based on the exercise data acquired in step S102, whether the target person P approaches the vehicle 2 at a speed equal to or higher than a predetermined threshold speed with his/her legs when approaching the vehicle 2 (S110).

Then, when it is determined in step S110 that the target person P approaches the vehicle 2 at the speed equal to or higher than the predetermined threshold speed by moving with his/her legs when approaching the vehicle 2 (S110, YES), the evaluation unit 52 determines that the health condition of the target person P is good (S112), and the setting unit 53 instructs the support device 5 of the vehicle 2 to apply the first setting to the operation of the vehicle 2 (S114).

Thereafter, the evaluation unit 52 determines whether the heart rate of the target person P decreases within a predetermined range until a predetermined time from the start of driving (S116). Then, when the heart rate of the target person P decreases within a predetermined range until a predetermined time from the start of driving (S116, YES), the evaluation unit 52 ends this processing. On the other hand, when the heart rate of the target person P does not decrease within a predetermined range until a predetermined time from the start of driving (S116, NO), the evaluation unit 52 determines that the health condition of the target person P is poor (S118), and the setting unit 53 instructs the support device 5 of the vehicle 2 to apply the second setting to the operation of the vehicle 2 (S120).

Subsequently, the notification unit 54 performs the second notification to the target person P (S128), and the information transmission unit 55 instructs the support device 5 to start regular transmission of the position information of the vehicle 2 (S130), and this processing ends.

On the other hand, when it is determined in step S110 that the target person P does not approach the vehicle 2 at the speed equal to or higher than the predetermined threshold speed by moving with his/her legs when approaching the vehicle 2 (S110, NO), the evaluation unit 52 determines that the health condition of the target person P is poor (S126), and the setting unit 53 instructs the support device 5 of the vehicle 2 to apply the second setting to the operation of the vehicle 2 (S128). Subsequently, the notification unit 54 performs the first notification to the target person P (S130), and the process proceeds to step S116. In this case, after the process proceeds to step S116, it is determined in a process of step S118 that the health condition of the target person is poor, as in step S126, and it is determined in a process of step S120 that the second setting is applied to the vehicle, as in step S128.

Here, steps S102, S104, S106, S110, S112, S116, S118, and S126 shown in FIG. 5 correspond to evaluation steps in the present disclosure. Further, steps S108, S114, S120, and S128 correspond to setting steps in the present disclosure.

### OTHER EMBODIMENTS

The vehicle 2 is shown as an example of a moving object in the above-described embodiment, but the moving object into which the target person P gets is not limited to the vehicle, and may be any moving object including ships and aircraft.

In the above-described embodiment, the acquisition unit 51 acquires the heart rate of the target person P from the wearable terminal 3 attached to the body of the target person P or the first heart rate sensor 9 provided on the door handle 7 of the vehicle 2, but a means for acquiring the heart rate is not limited thereto. For example, the acquisition unit 51 can acquire the heart rate of the target person P according to the related art from a facial image of the target person P around the vehicle 2 captured by the exterior camera 28 provided in the vehicle 2 or a facial image of the target person P inside the vehicle 2 captured by the interior camera 24. The images captured by the exterior camera 28 and the interior camera 24 can be acquired via the support device 5.

In addition, the acquisition unit 51 is assumed to calculate and acquire the ordinary heart rate based on the daily heart rate obtained from the wearable terminal 3, but the method of acquiring the ordinary heart rate is not limited thereto. For example, the acquisition unit 51 may acquire the ordinary heart rate input by the target person P to the mobile terminal 4 through the second touch panel 43. The target person P can input, for example, the ordinary heart rate measured at a fixed time every day at home or the ordinary heart rate measured at a medical facility during regular visits to the hospital, to the mobile terminal 4.

Some of the functional components of the mobile terminal 4 of the present embodiment may be distributed to other devices such as the support device 5 communicably connected to the mobile terminal 4. For example, the acquisition unit 51, the evaluation unit 52, the setting unit 53, the notification unit 54, and/or the information transmission unit 55 provided in the mobile terminal 4 shown in FIG. 3 may be provided in the support device 5.

Alternatively, the driver support system 1 includes an additional server, which is communicably connected to the mobile terminal 4 and the support device 5, for information processing, and the acquisition unit 51, the evaluation unit 52, the setting unit 53, the notification unit 54, and/or the information transmission unit 55 may be implemented in a computer of the additional server. Alternatively, the acquisition unit 51, the evaluation unit 52, the setting unit 53, the notification unit 54, and/or the information transmission unit 55 may be implemented by being distributed to the mobile terminal 4, the support device 5, and the additional server. In such a case, all of the first program 59 executed by the mobile terminal 4, the second program 79 executed by the support device 5, and the program executed by the computer of the additional server are equivalent to the driver support program in the present disclosure.

It should be noted that the present invention is not limited to the configuration of the above-described embodiment, and can be implemented in various forms without departing from the gist of the present invention.

### [Configurations Supported by Embodiment Described Above]

The above-described embodiment supports the following configurations.

(Configuration 1) A driver support system including: an acquisition unit that acquires a heart rate of a target person; an evaluation unit that evaluates a health condition of the target person based on the heart rate of the target person acquired by the acquisition unit; and a setting unit that performs an operation setting of a moving object into which the target person gets as a driver, the evaluation unit being configured to evaluate the health condition of the target person based on an ordinary heart rate, which is a heart rate of the target person during an ordinary period, and a pre-driving heart rate which is a heart rate of the target person when the target person arrives at the moving object, the setting unit being configured to perform the operation setting of the moving object based on a result of the health condition of the target person evaluated by the evaluation unit when the target person gets into the moving object as a driver.

According to the driver support system of Configuration 1, the health condition of the target person serving as the driver of the moving object is appropriately evaluated based on the ordinary heart rate and the pre-driving heart rate, the operation setting of the moving object is performed based on the evaluation result, whereby it is possible to provide appropriate support to the driver for safe driving.

(Configuration 2) In the driver support system according to Configuration 1, the evaluation unit determines that the health condition of the target person is good when a difference in heart rate obtained by subtracting the ordinary heart rate from the pre-driving heart rate is less than a predetermined threshold value, and determines that the health condition of the target person is poor when the difference in heart rate is equal to or higher than the predetermined threshold value.

According to the driver support system of Configuration 2, since the health condition of the target person P is evaluated based on the difference in heart rate between the ordinary heart rate and the pre-driving heart rate, it is possible to accurately evaluate the health condition of the target person P at the start of driving of the moving object.

(Configuration 3) In the driver support system according to Configuration 2, the acquisition unit acquires exercise data related to movement exercise of the target person when the target person approaches the moving object, and even when the difference in heart rate is equal to or higher than the predetermined threshold value, the evaluation unit determines that the health condition of the target person is good when determining, based on the exercise data, that the target person approaches the moving object at a speed equal to or higher than a predetermined threshold speed by moving with his/her legs.

According to the driver support system of Configuration 3, since a normal increase in heart rate due to the exercise state of the target person until arriving at the moving object (for example, an increase in heart rate due to running) cannot be treated as a sign of poor health, the health condition of the target person can be evaluated more appropriately.

(Configuration 4) In the driver support system according to Configuration 3, even when determining that the difference in heart rate is equal to or higher than the predetermined threshold value and the target person approaches the moving object at the speed equal to or higher than the predetermined threshold speed by moving with his/her legs, the evaluation unit determines that the health condition of the target person is poor when the heart rate of the target person does not decrease within a predetermined range until a predetermined time has elapsed from a start of driving of the moving object.

According to the driver support system of Configuration 4, since an unfavorable health condition can also be detected in which a recovery time of the heart rate, which has once increased, to the ordinary heart rate is longer than a predetermined time, the health condition of the target person can be evaluated more appropriately.

(Configuration 5) In the driver support system according to any one of Configurations 2 to 4, the setting unit applies a predetermined first setting to the operation setting of the moving object when the evaluation unit determines that the health condition of the target person is good, and applies a predetermined second setting, which is different from the first setting, to the operation setting of the moving object when the evaluation unit determines that the health condition of the target person is poor.

According to the driver support system of Configuration 5, it is possible to provide appropriate support to the target person for safe driving by switching the operation setting of the moving object according to the health condition of the target person at the start of driving.

(Configuration 6) In the driver support system according to any one of Configurations 2 to 5, the driver support system includes a notification unit that performs a notification to the target person who gets into the moving object, and the notification unit performs a first notification, which is a notification for driving support to the target person who is the driver of the moving object when the evaluation unit determines that the health condition of the target person is poor.

According to the driver support system 1 of Configuration 6, the target person cannot be bothered by unnecessary notifications when the health condition is good, and the target person can be given appropriate advice about driving of the moving object when the health condition is poor.

(Configuration 7) In the driver support system according to Configuration 6, the notification unit performs a second notification, which is a notification on the health condition, to the target person when the difference in heart rate is equal to or higher than the predetermined threshold value and the heart rate of the target person does not decrease within a predetermined range until a predetermined time elapses from a start of driving of the moving object.

According to the driver support system 1 of Configuration 7, when the health condition of the target person remains poor even after the start of driving, the target person can be notified of that effect to prompt the target person to take appropriate measures.

(Configuration 8) In the driver support system according to any one of Configurations 2 to 7, the driver support system includes an information transmission unit that transmits position information on a current position of the moving object to a server outside the moving object, and the information transmission unit transmits the position information of the moving object to the server at predetermined time intervals when the difference in heart rate is equal to or higher than the predetermined threshold value and the heart rate of the target person does not decrease within a predetermined range until a predetermined time elapses from a start of driving of the moving object.

According to the driver support system of Configuration 8, when the health condition of the target person remains poor even after the start of driving, the related person such as a family member or an acquaintance of the target person can access the server using own mobile terminal to trace whereabouts or movement routes of the target person, thereby watching movement of the target person.

(Configuration 9) In the driver support system according to any one of Configurations 1 to 8, the acquisition unit acquires the heart rate of the target person from a wearable terminal attached to a body of the target person.

According to the driver support system of Configuration 9, it is possible to easily acquire data on the ordinary hear rate of the target person during normal times. Further, since the heart rate is acquired using the wearable terminal that is worn on the body and can acquire biometric data in the vicinity of the body, the accuracy in measurement of a blood pressure value can be improved, and the health condition can be evaluated more accurately.

(Configuration 10) In the driver support system according to any one of Configurations 1 to 9, the acquisition unit acquires, from a heart rate sensor provided on a door handle of the moving object, a heart rate of the target person when the target person takes the door handle, and the evaluation unit uses the heart rate acquired by the acquisition unit from the heart rate sensor provided on the door handle, as the pre-driving heart rate of the target person.

According to the driver support system of Configuration 10, it is possible to reliably acquire the heart rate of the target person when the person arrives at the moving object.

(Configuration 11) In the driver support system according to Configuration 10, the setting unit limits some of functions of the moving object when the evaluation unit uses, as the pre-driving heart rate, the heart rate acquired from the heart rate sensor provided on the door handle, thereby determining that the health condition of the target person is poor.

According to the driver support system of Configuration 11, since the health condition of the target person can be grasped immediately before the target person gets into the moving object, when the target person whose health condition is poor gets into the moving object, the start of driving can be prevented, the operation of the moving object can be limited before the target person gets into the moving object, accidents can be prevented beforehand, and dangerous traveling can be avoided.

(Configuration 12) A driver support method executed by a computer, the driver support method including: an acquisition step of acquiring a heart rate of a target person; an evaluation step of evaluating a health condition of the target person based on the heart rate of the target person acquired in the acquisition step; and a setting step of performing an operation setting of a moving object into which the target person gets as a driver, the evaluation step including evaluating the health condition of the target person based on an ordinary heart rate, which is a heart rate of the target person during an ordinary period, and a pre-driving heart rate which is a heart rate of the target person when the target person arrives at the moving object, the setting step including performing the operation setting of the moving object based on a result of the health condition of the target person evaluated in the evaluation step when the target person gets into the moving object as a driver.

According to the driver support method of Configuration 12, the health condition of the target person serving as the driver of the moving object is appropriately evaluated based on the ordinary heart rate and the pre-driving heart rate, the operation setting of the moving object is performed based on the evaluation result, whereby it is possible to provide appropriate support to the driver for safe driving.

(Configuration 13) A driver support program causing a computer of a driver support system to function as: an acquisition unit that acquires a heart rate of a target person; an evaluation unit that evaluates a health condition of the target person based on the heart rate of the target person acquired by the acquisition unit; and a setting unit that performs an operation setting of a moving object into which the target person gets as a driver, the evaluation unit being configured to evaluate the health condition of the target person based on an ordinary heart rate, which is a heart rate of the target person during an ordinary period, and a pre-driving heart rate which is a heart rate of the target person when the target person arrives at the moving object, the setting unit being configured to perform the operation setting of the moving object based on a result of the health condition of the target person evaluated by the evaluation unit when the target person gets into the moving object as a driver.

According to the driver support program of Configuration 13, it is possible to realize the driver support system that can appropriately evaluate the health condition of the target person when the person gets into the moving object as a driver and provide the driver with appropriate support for safe driving in the moving object.

### REFERENCE SIGNS LIST

1 driver support system
2 vehicle
3 wearable terminal
4, 12 mobile terminal
5 support device
6 vehicle control device
7 door handle
8 touch sensor
9 first heart rate sensor
10 communication network
11 server
20 driver's seat
21 instrument panel
22 AV device
23 first touch panel
24 interior camera
25 speaker
26 microphone
27 door mirror
28 exterior camera
29 steering wheel
30 second heart rate sensor
31 seating sensor
40 first processor
41 first memory
42 first communicator
43 second touch panel
44 first GNSS sensor
45 acceleration sensor
50 arrival detection unit
51 acquisition unit
52 evaluation unit
53 setting unit
54 notification unit
55 information transmission unit
59 first program
60 second processor
61 second memory
62 second communicator
63 second GNSS sensor
70 communication control unit
71 image providing unit
72 sensor information providing unit
73 operation setting unit
74 AV control unit
75 position information transmission unit
79 second program.

What is claimed is:

1. A driver support system comprising a processor, wherein the processor includes:
an acquisition unit that acquires a heart rate of a target person;
an evaluation unit that evaluates a health condition of the target person based on the heart rate of the target person acquired by the acquisition unit; and
a setting unit that performs an operation setting of a moving object into which the target person gets as a driver,
the evaluation unit being configured to evaluate the health condition of the target person based on an ordinary heart rate, which is a heart rate of the target person during an ordinary period, and a pre-driving heart rate which is a heart rate of the target person when the target person arrives at the moving object,
the setting unit being configured to perform the operation setting of the moving object based on a result of the health condition of the target person evaluated by the evaluation unit when the target person gets into the moving object as a driver.

2. The driver support system according to claim 1, wherein
the evaluation unit determines that the health condition of the target person is good when a difference in heart rate obtained by subtracting the ordinary heart rate from the pre-driving heart rate is less than a predetermined threshold value, and determines that the health condition of the target person is poor when the difference in heart rate is equal to or higher than the predetermined threshold value.

3. The driver support system according to claim 2, wherein
the acquisition unit acquires exercise data related to movement exercise of the target person when the target person approaches the moving object, and
even when the difference in heart rate is equal to or higher than the predetermined threshold value, the evaluation unit determines that the health condition of the target person is good when determining, based on the exercise data, that the target person approaches the moving object at a speed equal to or higher than a predetermined threshold speed by moving with his/her legs.

4. The driver support system according to claim 3, wherein
even when determining that the difference in heart rate is equal to or higher than the predetermined threshold value and the target person approaches the moving object at the speed equal to or higher than the predetermined threshold speed by moving with his/her legs, the evaluation unit determines that the health condition of the target person is poor when the heart rate of the target person does not decrease within a predetermined range until a predetermined time has elapsed from a start of driving of the moving object.

5. The driver support system according to claim 2, wherein
the setting unit applies a predetermined first setting to the operation setting of the moving object when the evaluation unit determines that the health condition of the target person is good, and applies a predetermined second setting, which is different from the first setting, to the operation setting of the moving object when the evaluation unit determines that the health condition of the target person is poor.

6. The driver support system according to claim 2, wherein the processor comprising
a notification unit that performs a notification to the target person who gets into the moving object, wherein
the notification unit performs a first notification, which is a notification for driving support to the target person who is the driver of the moving object when the evaluation unit determines that the health condition of the target person is poor.

7. The driver support system according to claim 6, wherein
the notification unit performs a second notification, which is a notification on the health condition, to the target person when the difference in heart rate is equal to or higher than the predetermined threshold value and the heart rate of the target person does not decrease within a predetermined range until a predetermined time elapses from a start of driving of the moving object.

8. The driver support system according to claim 2, wherein the processor comprising
an information transmission unit that transmits position information on a current position of the moving object to a server outside the moving object, wherein
the information transmission unit transmits the position information of the moving object to the server at predetermined time intervals when the difference in heart rate is equal to or higher than the predetermined threshold value and the heart rate of the target person does not decrease within a predetermined range until a predetermined time elapses from a start of driving of the moving object.

9. The driver support system according to claim 1, wherein
the acquisition unit acquires the heart rate of the target person from a wearable terminal attached to a body of the target person.

10. The driver support system according to claim 1, wherein
the acquisition unit acquires, from a heart rate sensor provided on a door handle of the moving object, a heart rate of the target person when the target person takes the door handle, and
the evaluation unit uses the heart rate acquired by the acquisition unit from the heart rate sensor provided on the door handle, as the pre-driving heart rate of the target person.

11. The driver support system according to claim 10, wherein
the setting unit limits some of functions of the moving object when the evaluation unit uses, as the pre-driving heart rate, the heart rate acquired from the heart rate sensor provided on the door handle, thereby determining that the health condition of the target person is poor.

12. A driver support method executed by a computer, the driver support method comprising:
an acquisition step of acquiring a heart rate of a target person;
an evaluation step of evaluating a health condition of the target person based on the heart rate of the target person acquired in the acquisition step; and
a setting step of performing an operation setting of a moving object into which the target person gets as a driver,
the evaluation step including evaluating the health condition of the target person based on an ordinary heart rate, which is a heart rate of the target person during an ordinary period, and a pre-driving heart rate which is a heart rate of the target person when the target person arrives at the moving object,
the setting step including performing the operation setting of the moving object based on a result of the health condition of the target person evaluated in the evaluation step when the target person gets into the moving object as a driver.

13. A non-transitory computer-readable storage medium storing a driver support program which is executed by a computer of a driver support system, wherein the driver support program makes the computer function as:
an acquisition unit that acquires a heart rate of a target person;
an evaluation unit that evaluates a health condition of the target person based on the heart rate of the target person acquired by the acquisition unit; and
a setting unit that performs an operation setting of a moving object into which the target person gets as a driver,
the evaluation unit being configured to evaluate the health condition of the target person based on an ordinary heart rate, which is a heart rate of the target person during an ordinary period, and a pre-driving heart rate which is a heart rate of the target person when the target person arrives at the moving object,
the setting unit being configured to perform the operation setting of the moving object based on a result of the health condition of the target person evaluated by the evaluation unit when the target person gets into the moving object as a driver.
